(19) United States
(12) Patent Application Publication
(10) Pub. No.: US 2010/0041149 A1
(43) Pub. Date: Feb. 18, 2010

(54) BIOREACTOR AND METHOD FOR GENERATING CARTILAGE TISSUE CONSTRUCTS

(76) Inventors: Mark Weidenbecher, Cleveland, OH (US); Amad Awadallah, Cleveland, OH (US); James E. Dennis, Cleveland Heights, OH (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 12/541,680

(22) Filed: Aug. 14, 2009

Related U.S. Application Data
(60) Provisional application No. 61/089,238, filed on Aug. 15, 2008.

(51) Int. Cl. C12N 5/02; C12M 1/12

(52) U.S. Cl. 435/395; 435/297.1; 435/297.5

(57) ABSTRACT

A bioreactor includes a housing and a support mechanism for suspending the housing above the bottom surface of a culture vessel. The housing includes a member having oppositely disposed first and second surfaces and an inner surface defining an opening. The opening extends between the first and second surfaces of the member. The bioreactor includes a gas and liquid permeable membrane having first and second surfaces attached to the second surface of the member. The first surface of the gas and liquid permeable membrane and the member define a culture space for growing or culturing cells.

### RELATED APPLICATION

This application claims priority from U.S. Provisional Application No. 61/089,238, filed Aug. 15, 2008, the subject matter, which is incorporated herein by reference.

### TECHNICAL FIELD

The present invention relates generally to a device and method for generating tissue constructs, and more particularly to a bioreactor and method for generating cartilage tissue constructs comprised of chondrogenic cells dispersed within an endogenously produced extracellular matrix.

### BACKGROUND OF THE INVENTION

Articular cartilage has a minimal ability to heal and therefore has a tendency to accumulate damage over time. Accumulated minor damage, normal wear, and major damage often result in severely damaged cartilage that no longer provides structural support and results in significant pain and/or loss of joint movement. Interventional options for treating damaged cartilage typically include non-steroidal anti-inflammatory drugs, injection of hyaluronic acid, dietary changes, exercise, and, if the condition worsens, surgical intervention. Surgical intervention may include debridement, mosaiplasty, microfracture, and methods that use tissue engineering principles to repair damaged cartilage. If these interventions fail, the final option is often total joint arthroplasty.

When using tissue engineering methods, for example, a source of cartilage is needed. Typical sources of cartilage include mesenchymal stem cells and chondrocytes isolated from different parts of the body. Once isolated, these sources of chondrocytic cells need to be maintained and/or expanded in culture to obtain cell numbers sufficient for repair. Several methods are known that describe the use of serum-containing medium for cell expansion; however, the potential for these cells to differentiate into cartilage is unclear. Some reports indicate that chondrocytes quickly lose chondrogenic potential with passing in culture, while others describe specific culture conditions where expanded chondrocytes retain differentiation potential.

Once expanded, chondrogenic cells need to be delivered to a matrix that promotes, guides, and adheres the cells to a repair site. While the technology exists to prepare cartilage occupying a small area (e.g., approximately 1 cm in diameter), there is no described method to reproducibly produce cartilage tissue having a larger (i.e., greater than 2-5 cm) diameter and a thickness greater than 2 mm.

### SUMMARY OF THE INVENTION

The present invention relates generally to a device and method for generating tissue, and more particularly to a bioreactor and method for generating cartilage tissue constructs comprised of chondrogenic cells dispersed within an endogenously produced extracellular matrix.

According to one aspect of the present invention, a bioreactor can comprise a housing including a member having oppositely disposed first and second surfaces and an inner surface defining an opening. The opening can extend between the first and second surfaces of the member. The bioreactor can also include a gas and liquid permeable membrane having first and second surfaces attached to the second surface of the member. The first surface of the gas and liquid permeable membrane and the member can define a culture space for growing or culturing cells. Additionally, the bioreactor can include a culture vessel capable of receiving the housing and having a volume defined by a bottom surface and at least one side wall. The culture vessel can include a serum-free culture medium. The bioreactor can further include a support mechanism for suspending the housing above the bottom surface of the culture vessel so that the serum-free culture medium can contact the second surface of the gas and fluid permeable membrane.

According to another aspect of the present invention, a method for generating a cartilage tissue construct can comprise isolating a population of chondrogenic cells, expanding the population of chondrogenic cells, and then seeding the population of chondrogenic cells into a bioreactor. The bioreactor can comprise a housing including a member having oppositely disposed first and second surfaces, an inner surface defining an opening extending between the first and second surfaces, a gas an liquid permeable membrane having first and second surfaces attached to the second surface of the member, the first surface of the gas and liquid permeable membrane and the member defining a culture space for growing or culturing the population of chondrogenic cells, a culture vessel having a volume defined by a bottoms surface and at least one side wall and including a serum-free medium, and a support mechanism for suspending the housing above the bottom surface of the culture vessel. Next, the population of chondrogenic cells is cultured in the culture space of the bioreactor for a time sufficient to permit the population of chondrogenic cells to differentiate and form the cartilage tissue construct.

According to another aspect of the present invention, a cartilage tissue construct is provided. The cartilage tissue construct can comprise chondrogenic cells dispersed within an endogenously produced extracellular matrix.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a perspective view showing a bioreactor in an exploded configuration constructed in accordance with the present invention;

FIG. 2A is a perspective view showing the bioreactor in FIG. 1 in an assembled configuration;

FIG. 2B is a cross-sectional view taken from Line 2B-2B in FIG. 2A;

FIG. 3 is a perspective view showing a bottom portion of the bioreactor in FIG. 2A;

FIG. 4 is a perspective view showing a top portion of the bioreactor in FIG. 2A; and

FIG. 5 is a flow diagram illustrating a method for generating a cartilage tissue construct.

FIG. 6 (A-B) illustrates photographs showing T=native trachea, M=strap muscle flap, CS=engineered cartilage sheets, arrow=internal jugular vein and common carotid artery.

FIG. 7 (A-B) illustrates photographs showing tissue engineered trachea used for segmental tracheal reconstruction. M=pedicle of strap muscle flap, C=engineered neotrachel cartilage, NT=neotrachea, arrow=upper end-to-end anastomosis.

FIG. 8 illustrates photographs showing endoscopic (A) and macroscopic (B) view of a cicatricial stenosis within the neotracheal lumen 39 days following reconstruction.

### DETAILED DESCRIPTION

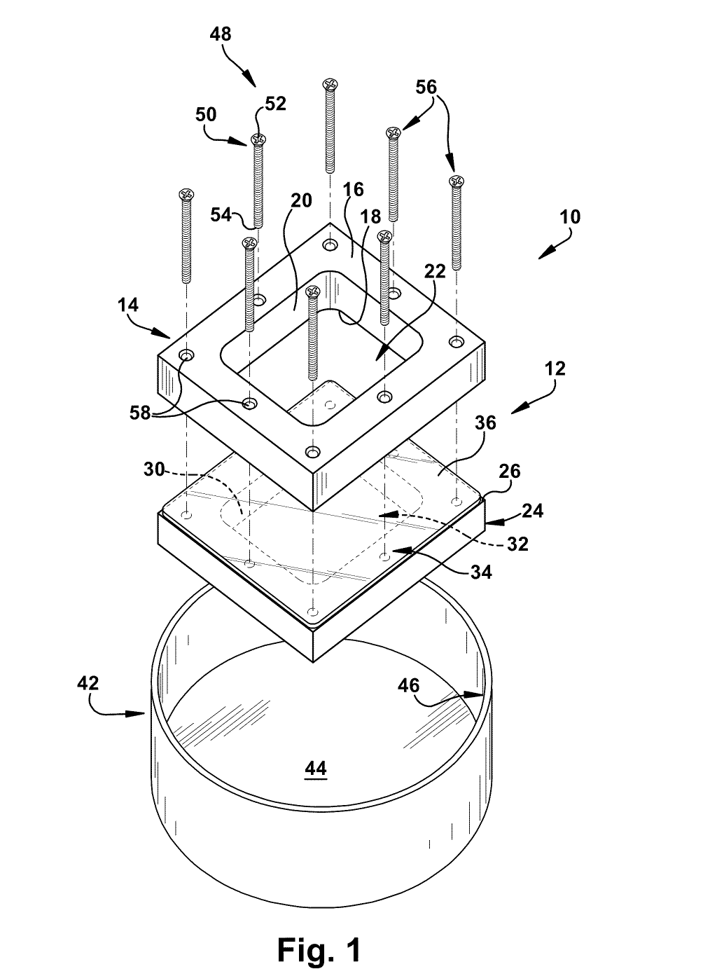

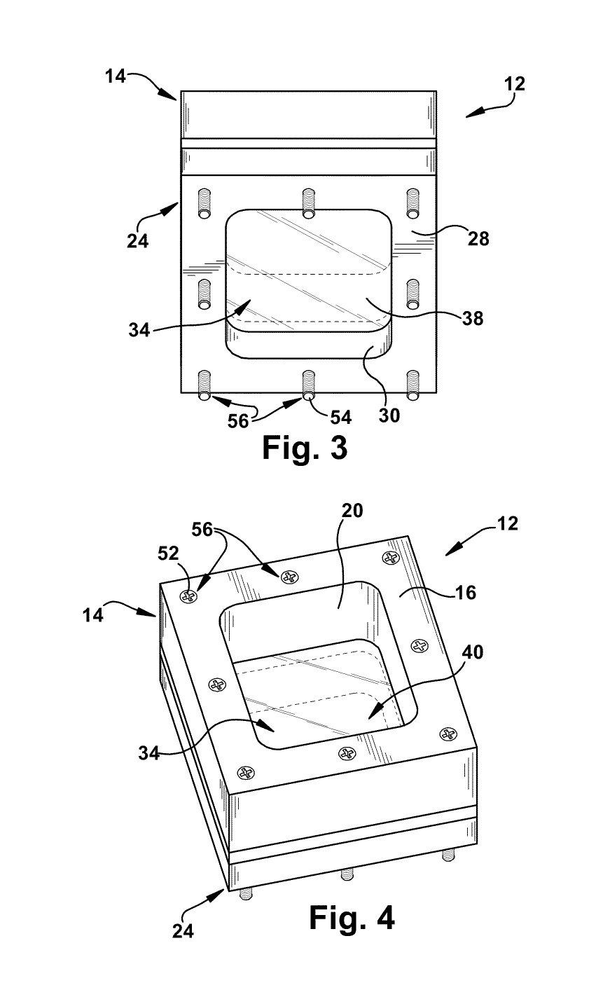

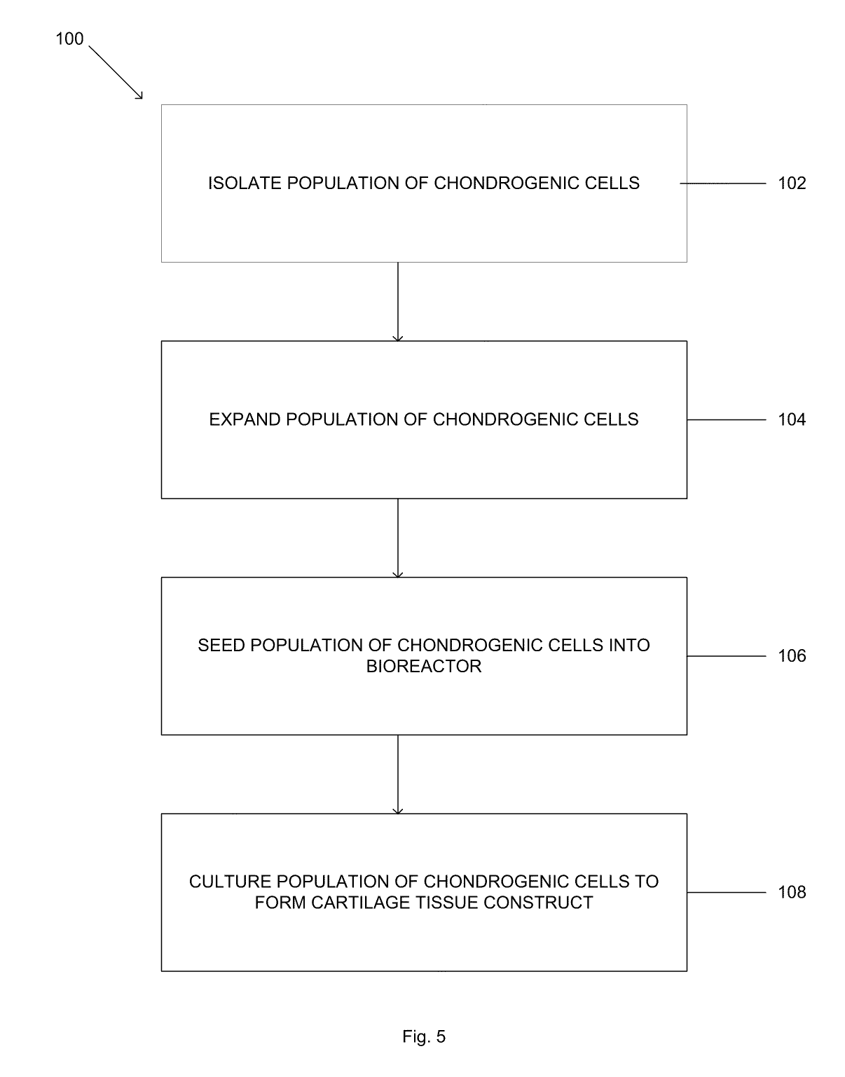

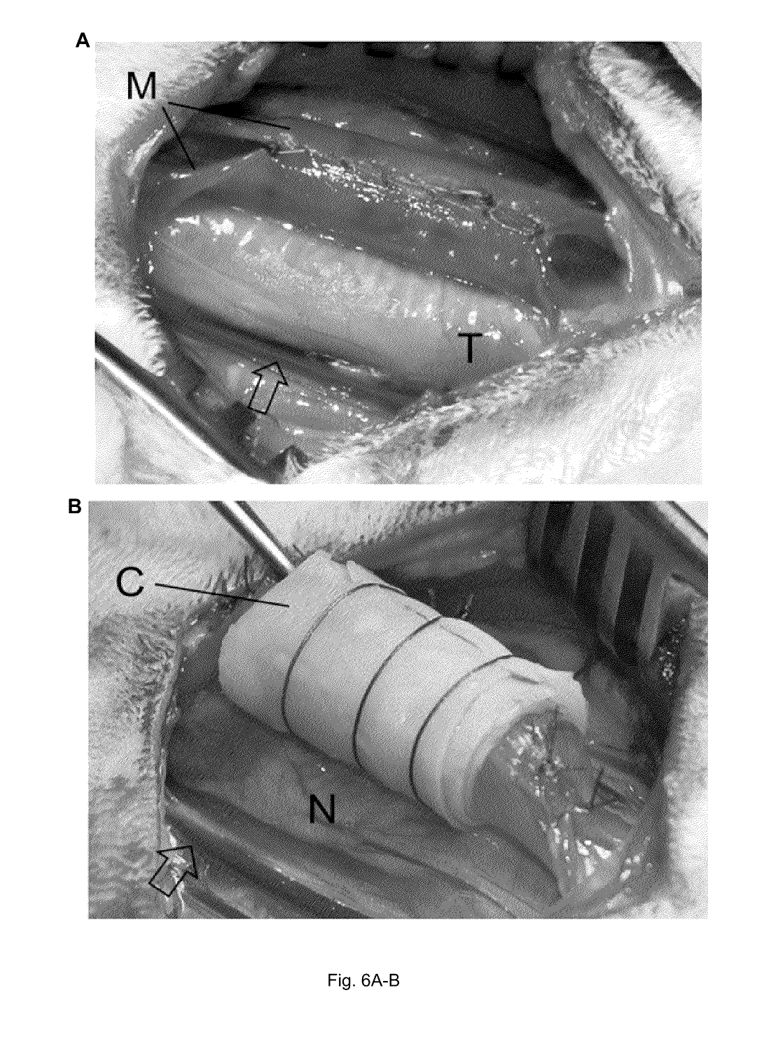

The present invention relates generally to a device and method for generating tissue constructs, and more particularly to a bioreactor and method for generating cartilage tissue constructs comprised of chondrogenic cells dispersed within an endogenously produced extracellular matrix. As representative of the present invention, FIGS. 1-4 illustrate a bioreactor 10 for culturing or growing chondrogenic cells to produce large, continuous sheets of cartilage having a thickness of about 200 microns to about 4 mm. The present invention also provides a method 100 (FIG. 5) for generating a cartilage tissue construct comprising a population of chondrogenic cells dispersed within an endogenously produced extracellular matrix. Although the present invention is described below in the context of generating a cartilage tissue construct, it will be appreciated that the present invention may be used to generate other types of tissue constructs including, for example, endothelial sheets, respiratory mucosa, myocardium, and skin.

All scientific and technical terms used in this application have meanings commonly used in the art unless otherwise specified. The definitions provided herein are to facilitate understanding of certain terms used frequently herein and are not meant to limit the scope of the present invention.

In the context of the present invention, the term “population” refers to an isolated culture comprising a homogenous, a substantially homogenous, or a heterogeneous culture of cells. Generally, a “population” may also be regarded as an “isolated” culture of cells.

As used herein, the term “chondrogenic cell” refers to any cell which, when exposed to appropriate stimuli, may differentiate and/or become capable of producing and secreting components characteristic of cartilage tissue.

As used herein, the term “cartilage” refers to a specialized type of dense connective tissue consisting of cells embedded in a matrix. There are several kinds of cartilage. Translucent cartilage having a homogeneous matrix containing collagenous fibers is found in articular cartilage, in costal cartilages, in the septum of the nose, in larynx and trachea. Articular cartilage is hyaline cartilage covering the articular surfaces of bones. Auricular cartilage is cartilage derived from the auricle of the ear. Costal cartilage connects the true ribs and the sternum. Fibrous cartilage contains collagen fibers. Yellow cartilage is a network of elastic fibers holding cartilage cells, which is primarily found in the epiglottis, the external ear, and the auditory tube. Cartilage is tissue made up of extracellular matrix primarily comprised of the organic compounds collagen, hyaluronic acid (a proteoglycan), and chondrocyte cells, which are responsible for cartilage production. Collagen, hyaluronic acid, and water entrapped within these organic matrix elements yield the unique elastic properties and strength of cartilage.

As used herein, the term “hyaline-like” refers to a type of cartilage known as hyaline cartilage. Hyaline cartilage includes the connective tissue covering the articular joint surface and may include, for example, articular cartilage, costal cartilage, auricular cartilage, and nose cartilage.

As used herein, the term “autologous” refers to cells or tissues that are obtained from a donor and then re-implanted into the same donor.

As used herein, the term “allogeneic” refers to cells or tissues that are obtained from a donor of one species and then used in a recipient of the same species.

As used herein, the term “construct” refers to a physical structure with mechanical properties, such as a matrix or scaffold.

As used herein, the term “mature chondrocyte” refers to a differentiated cell involved in cartilage formation and repair. Mature chondrocytes can include cells that are capable of expressing biochemical markers characteristic of mature chondrocytes, including, but not limited to, collagen type II, chondroitin sulfate, keratin sulfate, and characteristic morphologic markers including, but not limited to, rounded morphology observed in culture and in vitro generation of tissue or matrices with properties of cartilage.

As used herein, the term “immature chondrocyte” refers to any cell type capable of developing into a mature chondrocyte, such as a differentiated or undifferentiated chondrocyte as well as mesenchymal stem cells that can potentially differentiate into a chondrocyte Immature chondrocytes can include cells that are capable of expressing biochemical and cellular markers characteristic of immature chondrocytes, including, but not limited to, type I collagen, cathepsin B, modifications of the cytoskeleton, and formation of abundant secretory vesicles.

As used herein, the term “tracheal cartilage defect” refers to any tracheal defect of, or injury to, the trachea. Tracheal cartilage defects may be caused by a variety of factors including, but not limited to, stenosis caused by implanted prosthetic devices, penetrating or blunt trauma, and tumors. Additionally, tracheal cartilage defects may be caused by congenital defects ranging from the complete absence of the trachea to an incomplete or malformed trachea.

In an aspect of the present invention, a bioreactor 10 can comprise a housing 12 which includes a member 14 having oppositely disposed first and second surfaces 16 and 18 and an inner surface 20 defining an opening 22. As shown in FIG. 1, the opening 22 can extend between the first and second surfaces 16 and 18 of the member 14. The member 14 can have a thickness T, defined by the first and second surfaces 16 and 18. The member 14 can have a substantially square-shaped configuration as shown in FIG. 1; however, it will be appreciated that the member can have any other desired configuration, such as a substantially circular or rectangular shape, for example. The member 14 can be made from any one or combination of known materials. The materials can be biocompatible and can include, for example metals or metal alloys (e.g., stainless steel or titanium), hardened plastics (e.g., polyvinyl chloride), or glass.

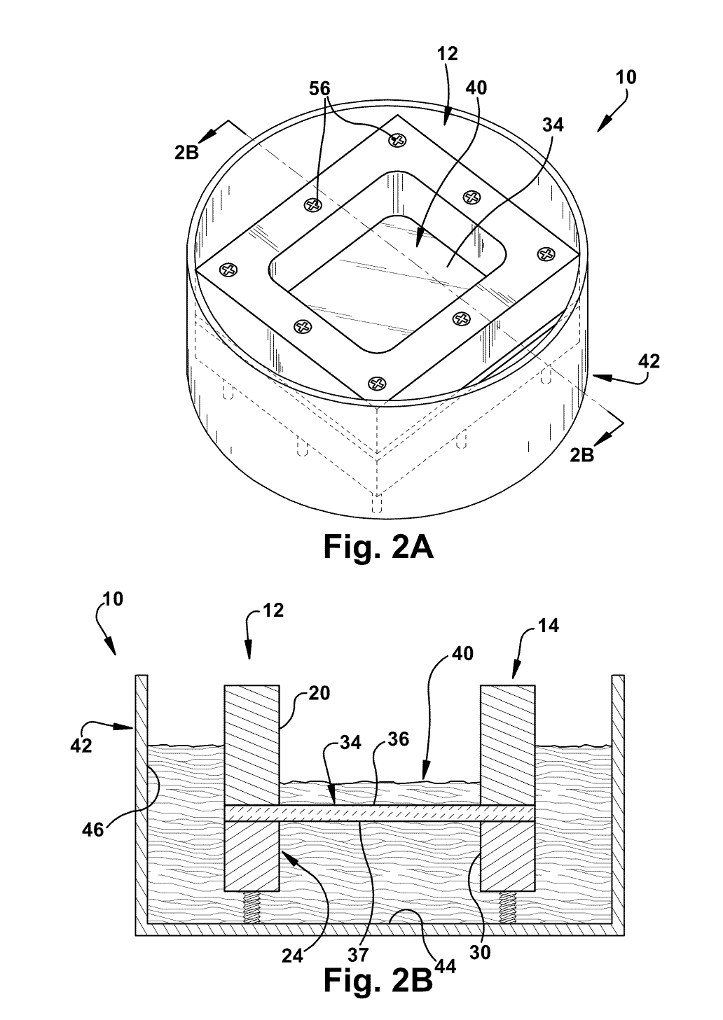

The housing 12 can additionally include a second member 24 having a configuration substantially identical to the configuration of the member 14. As shown in FIGS. 1-2B, for example, the second member 24 can have oppositely disposed first and second surfaces 26 and 28 and an inner surface 30 defining an opening 32. The opening 32 can extend between the first and second surfaces 26 and 28 of the second member 24. The second member 24 can have a thickness T₂ defined by the first and second surfaces 26 and 28. As shown in FIGS. 1-2B, the second member 24 can have a thickness T₂ that is less than the thickness T₁ of the member 14. The second member 24 can have a substantially square-shaped configuration having dimensions that are substantially identical to the dimensions of the member 14. The second member 24 can be made from any one or combination of known materials. The materials can be biocompatible and can include, for example metals or metal alloys (e.g., stainless steel or titanium), hardened plastics (e.g., polyvinyl chloride), or glass.

The bioreactor 10 can further include a gas and liquid permeable membrane 34 having first and second surfaces 36 and 38. As shown in FIG. 2B, the gas and liquid permeable membrane 34 can be coupled to the second surface 18 of the member 14. The first surface 36 of the gas and liquid permeable membrane 34 and the inner surface 20 of the opening 22 define a culture space 40 for growing or culturing cells. The gas and liquid permeable membrane 34 can serve as a substrate for cell attachment during culture. The gas and liquid permeable membrane 34 can have any desired configuration to facilitate gas and liquid perfusion therethrough. For example, the gas and liquid permeable membrane 34 can have a planar configuration with a pore size of about 5 microns and a thickness of about 10 microns. Alternatively, the gas and liquid permeable membrane 34 can have an arcuate shape to accommodate increases in pressure at the first surface 36 of the gas and liquid permeable membrane due to, for example, increase in cell density. Such a shape may prevent or mitigate sagging or tearing of the gas and liquid permeable membrane 34.

A number of different materials can be used to form the gas and liquid permeable membrane 34. Examples of such materials can include, but are not limited to, GORETEX, nylon (polyamides), DACRON (polyesters), polystyrene, polypropylene, polyacrylates, polyvinyl compounds (e.g., polyvinyl chloride), polycarbonates, polytetrafluoroethylene, TEFLON, THERMANOX, nitrocellulose, cotton, polyglycolic acid, cellulose, dextran, gelatin, etc.

As shown in FIGS. 1 and 2A, the bioreactor 10 can include a culture vessel 42 capable of receiving the housing 12. The culture vessel 42 can have a volume defined by a bottom surface 44 and at least one side wall 46. For example, the culture vessel 42 can include a 100 mm Petri dish. As described in more detail below, the culture vessel 42 can also include a serum-free medium for growing or culturing cells.

The bioreactor 10 can further include a support mechanism 48 for suspending the housing 12 above the bottom surface 44 of the culture vessel 42. By suspending the housing 12 above the bottom surface 44 of the culture vessel 42, the support mechanism 48 permits the serum-free culture medium to contact the second surface 38 of the gas and liquid permeable membrane 34. The support mechanism 48 additionally provides a space between the housing 12 and the bottom surface 44 in which a stir bar (not shown), for example, can be placed to facilitate circulation of the serum-free medium throughout the culture vessel 42.

The support mechanism 48 can comprise a variety of devices and can have any number of configurations. For example, the support mechanism 48 can include at least one member 50 having a first end 52 and a second end 54. The second end 54 of the at least one member 50 can contact the bottom surface 44 of the culture vessel 40 to support the housing 12 so that the serum-free medium can contact the second surface 38 of the gas and liquid permeable membrane 34. For example, the at least one member 50 can comprise a plurality of screws 56 capable of mating with a plurality of receptacle channels 58 extending between the first and second surfaces 16 and 18 of the member 14. The screws 56 can have a length longer than both the thickness T₁ of the member 14 and the thickness T₂ of the second member 24 so that the second end 54 of each of the screws, once inserted into the receptacle channels 58, can contact the bottom surface 44 of the culture vessel 42 and suspend the housing 12 above the bottom surface.

FIG. 5 is a flow diagram illustrating a method 100 for generating a cartilage tissue construct in accordance with another aspect of the present invention. In the method 100, a population of chondrogenic cells may be isolated at 102. Chondrogenic cells may be isolated from essentially any tissue by obtaining, for example, a tissue biopsy. Chondrogenic cells may be isolated directly from pre-existing cartilage tissue such as hyaline cartilage, elastic cartilage, or fibrocartilage. More specifically, chondrogenic cells may be isolated from articular cartilage (from either weight-bearing or non-weight-bearing joints), costal cartilage, nasal cartilage, auricular cartilage, tracheal cartilage, epiglottic cartilage, thyroid cartilage, arytenoid cartilage, and/or cricoid cartilage. Alternatively, chondrogenic cells may be isolated from bone marrow or an established cell line.

Chondrogenic cells may be allogeneic, autologous, or a combination thereof, and may be obtained from various biological sources. Biological sources may include, for example, both human and non-human organisms. Non-human organisms contemplated by the present invention include primates, livestock animals (e.g., sheep, pigs, cows, horses, donkeys), laboratory test animals (e.g., mice, hamsters, rabbits, rats, guinea pigs), domestic companion animals (e.g., dogs, cats), birds (e.g., chicken, geese, ducks, and other poultry birds, game birds, emus, ostriches), captive wild or tamed animals (e.g., foxes, kangaroos, dingoes), reptiles and fish.

After obtaining a tissue biopsy of auricular cartilage, for example, the chondrogenic cells may be released by contacting the tissue biopsy with at least one agent capable of dissociating the chondrogenic cells. Examples of agents that can be used include trypsin and collagenase enzymes. As illustrated in Example 1 of the present invention, for example, a tissue biopsy may be sequentially digested in about 0.25% trypsin/EDTA for about 30 minutes, about 0.1% testicular hyaluronidase for about 15 minutes, and about 0.1% collagenase type II for about 24 hours. The digestion may be carried out at about 37° C. in about a 20 ml volume. Any undigested tissue and/or debris can be removed by filtering the cell suspension using a Nitex 70 μm sterile filter followed by centrifugation. The viability of the cells can be assessed by Trypan Blue dye exclusion test. By digesting the tissue biopsy, a population of chondrogenic cells comprising mature chondrocytes, immature chondrocytes, or a combination thereof, may be successfully isolated from the tissue biopsy.

The isolated population of chondrogenic cells may next be expanded at 104 in a conditioned growth media effective to promote expansion of the cells. For example, once the chondrogenic cells have been isolated from the tissue biopsy, they may be proliferated ex vivo in monolayer culture using conventional techniques well known in the art. Briefly, the chondrogenic cells may be passaged after the cells have proliferated to such a density that they contact one another on the surface of a cell culture plate. During the passaging step, the cells may be released from the substratum. This may be performed by routinely pouring a solution containing a proteolytic enzyme, such as trypsin, onto the monolayer. The proteolytic enzyme hydrolyzes proteins which anchor the cells on the substratum and, as a result, the cells may be released from the surface of the substratum. The resulting cells may now be in suspension, diluted with culture medium, and re-plated into a new tissue culture dish at a cell density such that the cells do not contact one another. The cells subsequently re-attach onto the surface of the tissue culture and start to proliferate once again. Alternatively, the cells in suspension may be cryopreserved for subsequent use using techniques well known in the art.

In an example of the method 100, a population of immature chondrocytes may be expanded ex vivo in a conditioned growth media at a desired density. More particularly, the isolated cells can be counted, plated at densities not greater than about 5,700 cells/cm², and expanded in growth media (Dulbecco's Modified Eagle's Medium, DMEM) with about 1 g/L glucose supplemented with about 10% fetal bovine serum (FBS, Invitrogen, Carlsbad, Calif.) at about 37° C. in a humidified atmosphere of about 95% air and about 5% carbon dioxide. Media can be changed every three to four days, or as needed. When cell cultures reach confluence, they can be typsinized, frozen in expansion medium containing 10% dimethyl sulfoxide (Sigma-Aldrich, St. Louis, Mo.), and stored in liquid nitrogen. Prior to culture in the bioreactor 10, the cells can be thawed and expanded for two additional passages. The medium may be replaced twice per week, for example, and confluent plates may be passaged as needed to obtain a desired cell density. In one example of the invention, the immature chondrocytes can include mesenchymal stem cells that are expanded in an expansion media that includes FGF.

Either prior to or after expanding the population of cells at 104, the bioreactor 10 can be assembled as follows. First, the member 14 and the second member 24 of the housing 12 can be placed in a sterilization solution, such as a 90% ethanol solution, for about 10 minutes and then sonicated. After sterilizing the housing 12, a gas and liquid permeable membrane 34 comprised of polyester, for example, can be placed on the first surface 26 of the second member 24. Next, the second surface 18 of the member 14 can be contacted with the first surface 36 of the gas and liquid permeable membrane 34 so that the gas and liquid permeable membrane is sandwiched between the member and the second member 24.

Using tweezers or a hole punch, for example, holes can then be punched into the gas and liquid permeable membrane 34 to allow insertion of screws 56 into the receptacle channels 58. While maintaining the gas and liquid permeable membrane 34 under slight tension, the screws 56 can be carefully threaded into the receptacle channels 58 to avoid wrinkling or waviness of the gas and liquid permeable membrane. Any areas where the gas and liquid permeable membrane 34 protrudes out of the housing 12 can be trimmed using a scalpel, for example. The assembled housing 12 can be placed into a culture vessel 42, such as a 100 mm Petri dish, autoclaved, and then stored until needed.

After preparing the bioreactor 10, about 50 ml of a serum-free culture medium can be added to the culture vessel 42. The serum-free medium may comprise, for example, high-glucose DMEM supplemented with dexamethasone, ascorbate-2-phosphate, sodium pyruvate, and a premix of insulin, transferrin and selenium (ITS). More particularly, and as described in Example 1, the serum-free medium may comprise high-glucose DMEM containing about 100 mM sodium pyruvate, about 80 μM ascorbate-2-phosphate, about 100 nM dexamethasone, and about 1% ITS. Additional serum-free medium components may include L-Glutamine, MEM non-essential amino acid solution, and/or an antibiotic/antimycotic.

It should be appreciated that growth factors may also be added to the serum-free medium to enhance or stimulate cell growth. Examples of growth factors include, but are not limited to, transforming growth factor-β, platelet-derived growth factor, insulin-like growth factor, acid fibroblast growth factor, basic fibroblast growth factor, epidermal growth factor, hepatocytic growth factor, keratinocyte growth factor, and bone morphogenic protein. It should also be appreciated that other agents, such as cytokines, hormones (e.g., parathyroid hormone, parathyroid hormone-related protein, hydrocortisone, thyroxine, insulin) and/or vitamins (e.g., vitamin D) may also be added or removed from the serum-free medium to promote cell growth. In one example, the growth factor added to the medium can include an amount of transforming growth factor-β effective to promote differentiation of expanded mesenchymal stem cells.

The serum-free culture medium can be added to the culture vessel 42 so that the level of the medium rises above the level of the gas and liquid permeable membrane 34 without contacting the first surface 16 of the member 14 (i.e., without flowing over into the culture space 40). Raising serum-free medium above the level of the gas and liquid permeable membrane 34 can create a pressure differential between the first surface 36 of the gas and liquid permeable membrane and the surface of the medium. The pressure differential can cause some of the medium to perfuse through the gas and liquid permeable membrane 34 and into the culture space 40. If this occurs, the medium can be removed (e.g., aspirated) from the first surface 36 of the gas and liquid permeable membrane 34.

Next, the population of cells can be seeded or loaded into the bioreactor 10 at 106 using, for example, a syringe. More particularly, the cells can be delivered to the culture space 40 of the bioreactor 10. The cells should be carefully added to the culture space 40 so that the cells do not spill out of the culture space and into the culture vessel 42. Once loaded into the culture space 40, the cells can be cultured under appropriate conditions and the media changed as needed to facilitate formation of the cartilage tissue construct.

In an example of the method 100, about 8 ml to about 10 ml of a solution containing about 1×10⁶ to about 4×10⁷ cells can be dispensed into the culture space 40 using a syringe. The cells can then be cultured at about 37° C. in about a 5% carbon dioxide atmosphere at about 90% to about 95% humidity. The oxygen percentage can be varied from about 1% to about 21%. Growth media can be changed daily or as needed. Since it is particularly important that the medium level does not drop below the level of the gas and liquid permeable membrane 34, which may cause air bubble formation at the second surface 38 of the gas and liquid permeable membrane, expired medium can be removed from one portion of the culture vessel 42 while fresh medium is simultaneously added at another portion of the culture vessel.

At 108, the cells can be cultured for a time and under conditions sufficient to form a confluent cell monolayer at the first surface 36 of the gas and liquid permeable membrane 34. The confluency of the cells, as well as the thickness of the cell monolayer, can be assessed using microscopic means, for example. Once the cartilage tissue construct has a desired thickness, the cartilage tissue construct can be harvested as described below. If a cartilage tissue construct having a greater or increased thickness is desired, however, one or more additional administrations of cells can be delivered to the culture space 40 of the bioreactor 10. Each successive administration of cells can form a confluent cell monolayer over the pre-existing cell monolayer and thereby form a cartilage tissue construct having a thickness of between, for example, about 200 microns to about 4 mm.

After forming the cartilage tissue construct, sterile instruments can be used to remove the cartilage tissue construct from the bioreactor 10. For example, a scalpel can be used to remove the portion of the gas and liquid permeable membrane 34, which includes the cartilage tissue construct. Upon cutting the gas and liquid permeable membrane 34, the gas and liquid permeable membrane may sink to the bottom surface 44 of the culture vessel 42. The housing 12 can then be removed from the culture vessel 42 and the gas and liquid permeable membrane 34 retrieved. The gas and liquid permeable membrane 34 can then be carefully separated from the cartilage tissue construct using tweezers, for example. The cartilage tissue construct can be harvested for use or, as described above, can be further cultured to produce thicker cartilage tissue constructs.

The cartilage tissue construct produced by the method 100 of the present invention can find use in a variety of applications. One example of such an application can include forming a whole or partial portion of a trachea to treat a tracheal defect in a subject. A tracheal implant may be formed according to the method 100 of the present invention (as described above). Depending upon the clinical needs of the subject, a cartilage tissue construct may be used to form a whole trachea or only a portion of a whole trachea. For example, a tracheal implant comprising a whole trachea may be formed by first obtaining a tube-shaped tracheal construct comprised of, for example, a biocompatible and/or bioresorbable material. The tracheal construct may be optimally sized to suit the needs of the subject. The tissue construct may then be wrapped around the tracheal construct and secured with a fibrin sealant and/or sutures, for example. After the tracheal implant has been formed, the implant may be used to repair a tracheal cartilage defect as described in greater detail below.

In an example of the present invention, a population of chondrogenic cells may be isolated at 102 and expanded at 104 as described above. As also described above, the population of chondrogenic cells may then be seeded into a bioreactor 10 at 106 and cultured to form a cartilage tissue construct at 108. After forming the cartilage tissue construct, the cartilage tissue construct may be removed from the bioreactor 10 (as described above) and then formed into a tracheal implant.

Repair of a tracheal cartilage defect may begin by first identifying the defect. Tracheal cartilage defects may be readily identifiable by visually identifying the defects during open surgery of the trachea or, alternatively, by using computer aided tomography, X-ray examination, magnetic resonance imaging, analysis of serum markers, or by any other procedures known in the art.

Once the tracheal cartilage defect has been identified, an appropriately-sized tracheal implant may be selected. For example, the tracheal implant may have a size and shape so that when the tracheal implant is implanted, the edges of the tracheal implant directly contact the edges of native cartilage tissue. The tracheal implant may be fixed in place by, for example, surgically fixing the implant with bioresorbable sutures. Additionally or optionally, the tracheal implant may be fixed in place by applying a bioadhesive to the region interfacing the tracheal implant and the tracheal cartilage defect. Examples of suitable bioadhesives include fibrin-thrombin glues and synthetic bioadhesives similar to those disclosed in U.S. Pat. No. 5,197,973.

The cartilage tissue defect may comprise a stenotic portion of the trachea, such as two of the cartilages comprising the trachea, caused by prolonged placement of a tracheal T-tube. To repair the tracheal cartilage defect, the stenotic portion may first be surgically excised. Next, a tracheal implant may be formed having a size and shape complementary to the size and shape of the excised stenotic portion. The tracheal implant may then be surgically fixed in place of the excised stenotic portion by an end-to-end anastomosis. After the tracheal implant has been suitably fixed in place, the surgical procedure may be completed and the tracheal implant permitted to integrate into the native cartilage tissue.

In an alternative example of the present invention, the tracheal cartilage defect may comprise a congenital defect, such as a missing trachea, in a pediatric subject. A tracheal implant comprising a whole trachea may be prepared and then surgically implanted into the subject by an end-to-end anastomosis. After the tracheal implant has been suitably fixed in place, the surgical procedure may be completed and the tracheal implant permitted to integrate into the native tissue. By providing the subject with a whole tracheal implant, the tracheal implant may integrate into the native tissue and grow along with the subject, thus removing the need to perform additional surgeries as the subject ages.

As noted above, the present invention may also be used to form tissue constructs other than cartilage. By culturing different types of cells in the bioreactor 10, different types of tissue constructs can be formed. For example, seeding the bioreactor 10 with endothelial cells, myocytes, and/or skin precursor cells and then culturing the cells according to the present method can generate tissue constructs such as respiratory mucosa, myocardium, and/or skin (respectively).

It will be appreciated that the present invention can be used in a variety of applications besides transplantation or implantation of cartilage tissue constructs in vivo. Examples of other uses can include, but are not limited to, screening cytotoxic compounds, allergens, growth/regulatory factors, pharmaceutical compounds, etc. in vitro, elucidating the mechanisms of certain diseases, studying the mechanisms by which drugs and/or growth factors operate, diagnosing and monitoring cancer in a patient, gene therapy, and the production of biological products.

The following example is for the purpose of illustration only and is not intended to limit the scope of the claims, which are appended hereto.

### EXAMPLE 1

### Harvesting Cartilage, Isolating and Expanding Cells

Under sterile conditions, a 1×1 cm piece of auricular cartilage is harvested from the ears of New Zealand White rabbits (ages 9-15 months). The perichondrium is removed to avoid cell contamination and the cartilage samples are diced to approximately 1 mm³ pieces. The diced cartilage is digested sequentially for 15 minutes in 0.1% testicular hyaluronidase (261 U/ml 20 min, H-3506, Sigma Chemical Co, St. Louis, Mo.), 30 minutes in 0.25% trypsin/EDTA (Invitrogen, Carlsbad, Calif.) and 24 hours in 0.1% collagenase type II (422 U/ml, 24 hrs, CLS 2, Worthington, Lakewood, N.J.). All digestions are carried out at 37° C. in a 20 ml volume on an incubated rocker at 37° C. The undigested tissue and debris are removed by filtering the cell suspension using a 70 Mm sterile Nitex filter and the cell suspension then centrifuged. The viability of the chondrocytes is assessed by Trypan Blue dye exclusion test. The isolated cells are counted, plated at densities not greater than 5700 cells/cm², and expanded in growth media (Dulbecco's Modified Eagle's Medium, DMEM) with 1 g/L glucose supplemented with 10% fetal bovine serum (FBS, Invitrogen, Carlsbad, Calif.) at 37° C. in a humidified 95% air and 5% carbon dioxide atmosphere. Media is changed every 3 to 4 days. When cell cultures reach confluence, they are typsinized, frozen in expansion medium containing 10% dimethyl sulfoxide (Sigma-Aldrich, St. Louis, Mo.), and stored in liquid nitrogen. Prior to scaffold-free cartilage culture, the cells are thawed and expanded for two additional passages. At the end of the second passage following thaw, cells are placed into bioreactor culture.

### Production of Scaffold-Free Cartilage Sheets Using the Bioreactor

Defined medium consisting of DMEM with 4.0 g/L glucose supplemented with 1% ITS+Premix™ (BD Biosciences, San Jose, Calif.), 37.5 μg/mL ascorbate-2-phosphate (Wako Chemicals, Richmond, Va.), and 10⁻⁷ M dexamethasone (Sigma-Aldrich, St. Louis, Mo.) is used for bioreactor 10 culture. In addition, the media is supplemented with 2 mM L-glutamine, 10.000 U/ml penicillin G sodium, 10,000 μg/ml streptomycin sulfate, and 25 μg/ml amphotericin B in 0.85% saline, 1% nonessential amino acids and 1% sodium pyruvate (Invitrogen, Carlsbad, Calif.). The defined media encourages chondrocytes to switch from an expansion mode into a redifferentiation mode with production of extracellular cartilage matrix.

Prior to adding the cells into the culture space 40, 50 ml of media are added to the culture vessel 42 with no media being added to the culture space. As soon as the media level rises above the level of the gas and liquid permeable membrane 34, small amounts of media slowly start to diffuse through the membrane; this media is removed just prior to loading the cells into the culture space 40. The culture space 40 holds approximately 10 ml, but to avoid any spillage of cells from the culture space to the outside, cells are diluted in 8 ml of growth media and then added onto the gas and liquid permeable membrane 34. Several cell loading densities between 1.5×10⁷ and 3.5×10⁷ have been tested in the past. Because the culture space 40 of the bioreactor 10 is open, it is possible to add a second, third, or more cell layers onto the preexisting cartilage layer to result in an increased thickness of the cartilage.

Culture conditions are 37° C. in a 5% carbon dioxide atmosphere at 95% humidity, and growth media is changed daily until the cartilage is removed from the bioreactor 10. Since it is important that the fluid level does not drop below the level of the gas and liquid permeable membrane 34, which would allow forming an air bubble underneath the membrane (which is difficult to remove), the spent media is removed from one side of the bioreactor 10 while, at the same time, new media is added on the opposite side.

Harvesting the Engineered Cartilage from the Bioreactor

Sterile instruments are used to remove the cartilage sheet from the bioreactor 10. A scalpel is used to cut along the edge of the bioreactor 10 to remove the entire gas and liquid permeable membrane 34 together with the cartilage layer attached to it. As soon as the membrane 34 is completely detached from the housing 12, it sinks. The housing 12 can now be removed from the culture vessel 42. Next, the gas and liquid permeable membrane 34 is carefully separated from the cartilage sheets using tweezers. Following this step, the cartilage can be harvested or further cultured in growth media which has shown to produce even thicker cartilage sheets with greater amounts of cartilage matrix.

From the above description of the invention, those skilled in the art will perceive improvements, changes and modifications. Such improvements, changes and modifications within the skill of the art are intended to be covered by the appended claims.

### EXAMPLE 2

### Use of Bioreactor to Prepare Tissue-Engineered Trachea for Airway Reconstruction

We developed a technique to engineer scaffold free cartilage, a biocompatible, autologous neotracheal constructs in rabbits. The constructs, which were implanted up to 12 months, formed a vascularized tracheal substitute with excellent rigidity and flexibility very similar to the mechanical properties of a rabbit's native trachea.

Using a similar tracheal engineering approach, we determined neotracheal suitability and functionality for segmental tracheal reconstruction in rabbits.

### Material and Methods

### Cell Culture

Six New Zealand White adult male rabbits, weighing 3.0-3.5 kg and 12 to 14 months of age, were used to harvest a 5×5 mm piece of auricular cartilage under sterile conditions. The perichondrium was carefully removed to minimize potential contamination with fibroblastic cells. The cartilage was cut into approximately 1 mm3 pieces, sequentially digested and expanded in culture as previously described. Medium was replaced every 3 to 4 days, confluent plates were trypsinized, the cells frozen in expansion medium containing 10% dimethyl sulfoxide (Sigma-Aldrich, St. Louis, Mo.) and stored in liquid nitrogen for following experiments. Fabrication of scaffold-free cartilage sheets

Prior to the bioreactor culture, the chondrocytes were thawed, seeded at 5,000 cells/cm2 and expanded in 175 cm2 culture flasks. Non-adherent cells were removed by changing growth media after 4 days. Cells were passaged by standard methods using trypsin after reaching confluence and subcultured. Chondrocytes from second passage were used to form scaffold-free engineered cartilage. Expanded cells were counted and 5.0×107 cells resuspended in 15 ml of chondrogenic defined medium loaded into a custom designed bioreactor, which allowed fabrication of 1 mm thick scaffold-free cartilage sheets. Briefly, the bioreactor is a 4×4 cm semi-open chamber holding a gas and water permeable membrane. Cells were applied onto the membrane, where they started to form a coherent cell layer, which over the course of 4 weeks, developed a cartilage sheet. Culture medium was changed every other day. The medium consisted of Dulbecco's Minimal Essential Medium with 4.0 g/L glucose supplemented with 1% ITS+Premix™ ([BD Biosciences, San Jose, Calif.], 37.5 μg/mL ascorbate-2-phosphate [Wako Chemicals, Richmond, Va.], and 10-7 M dexamethasone [Sigma-Aldrich, St. Louis, Mo.]). In addition, the media contained 2 mM L-glutamine, antimycotic-antibacterial supplements (10.000 U/ml penicillin G sodium, 10.000 μg/ml streptomycin sulfate, and 25 μg/ml amphotericin B in 0.85% saline), nonessential amino acids and sodium pyruvate (Invitrogen, Carlsbad, Calif.), each at 1%. After 4 weeks, the cartilage sheet was peeled off the bioreactor membrane and transferred into a 150 mm large culture dish, where they were allowed to float freely. After an additional 4 weeks in the dish, the sheets had reached a thickness of approximately 1 mm with adequate strength for neotracheal fabrication.

### Surgical Procedure

### Neotracheal Fabrication

A total of six New Zealand White rabbits received a neotracheal construct each, which was implanted paratracheally into the neck. The protocol was approved by the animal care committee of Case Western Reserve University of Cleveland. The rabbits were anesthetized by an intramuscular injection of ketamine hydrochloride (70 mg/kg) and xylazine (7 mg/kg). Their necks were shaved and disinfected with 10% povidone-iodine and 70% ethanol and positioned supine. A midline neck incision was made, strap muscles were identified, a bipedicled muscle flap consisting of both sternohyoid muscles raised, wrapped around a 7.5 mm wide and 30 mm long sterile silicone tube and secured with 5-0 Vicryl sutures (FIG. 6). Three layers of autologous scaffold free cartilage, each 1 mm thick, were wrapped around the silicone-muscle construct leaving a gap at the posterior aspect and secured with 4-0 Vicryl creating a 3 layered construct consisting of a silicone tube, muscle flap and engineered cartilage. The bipedicled, sternohyoid muscle flap with its blood supply form superior and inferior served as a vessel carrier for the neotrachea's intrinsic vascularisation. The construct was placed paratracheally and the neck was closed using absorbable sutures. Postoperatively, the animals were observed for approximately 2 hours before being returned to their cages, where water and standard feed were available. For the following 3 days, the rabbits were given 10 mg/kg enrofloxacin as a prophylaxis. The animals were monitored for signs of infection and were weighed weekly.

### Tracheal Reconstruction

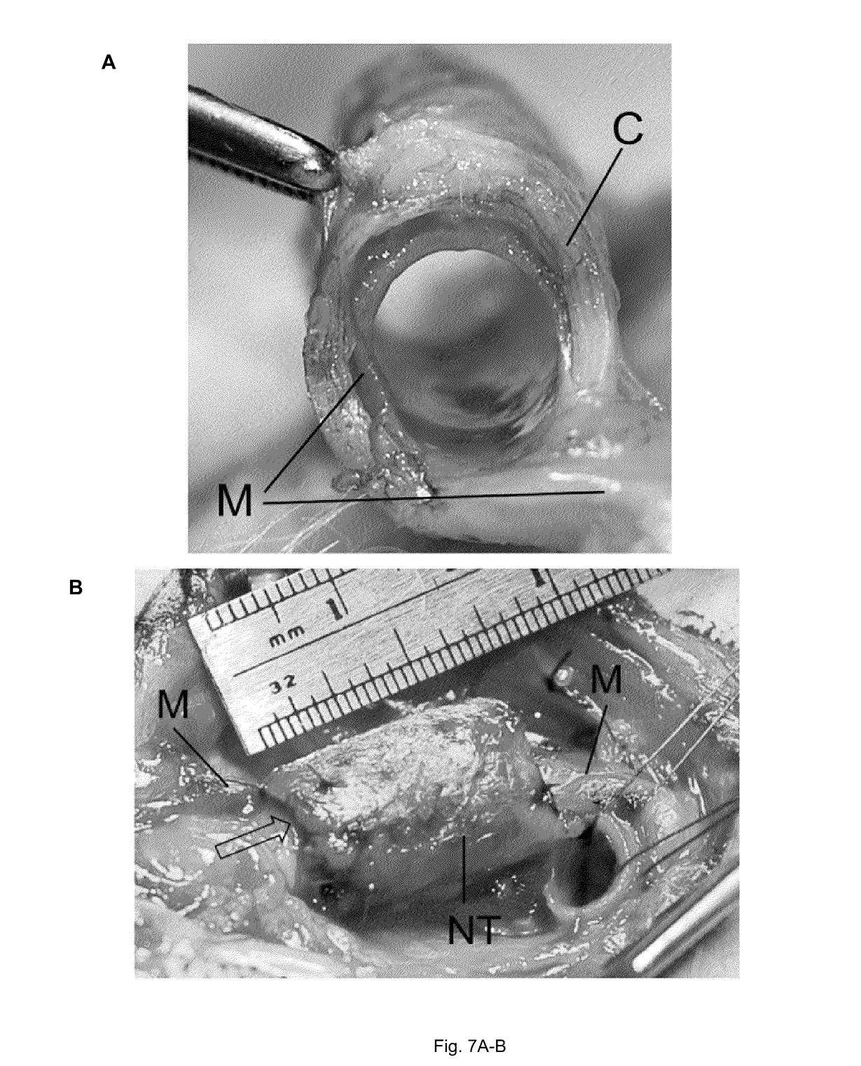

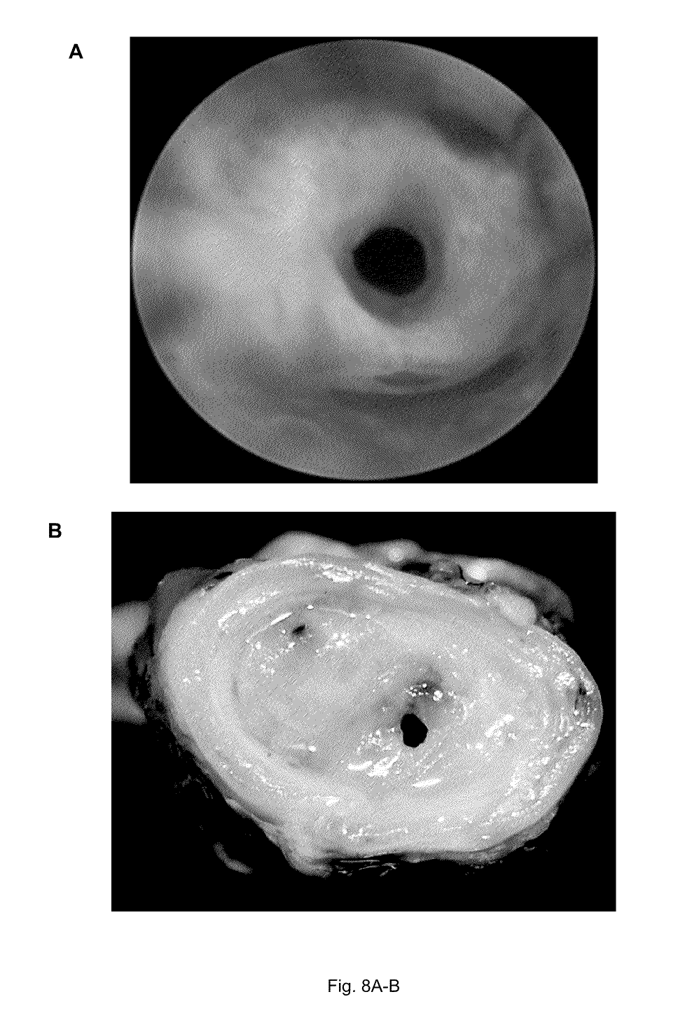

Segmental tracheal reconstruction was performed 12-14 weeks following neotracheal implantation. A group of 2 rabbits underwent tracheal reconstruction at a time to allow modifying the surgical technique if needed. For segmental tracheal reconstruction, the rabbits were anesthetized and prepped as previously described. A midline incision was made, and trachea and neotrachea identified and exposed. The silicone tube was removed and neotracheas manually assessed for adequate mechanical stability prior reconstruction. A 2 cm long segment of the cervical, native trachea was resected after identifying the laryngeal recurrent nerve. Care was taken not to penetrate the esophagus. In order to prevent muscle flap compression at the tracheal end-to-end anastmosis, the diameter of the neotracheal framework was designed 1 mm larger than the diameter of the native trachea. Upper and lower end-to-end anastomosis was done by using 4-0 Vicryl (FIG. 7). All knots were placed extraluminally to minimize granulation tissue. An airtight anastomosis was ensured and the wound closed with 4-0 vicryl sutures in layers. Rabbits were recovered as described above and antibiotics given as described above. In addition, the animals received a daily dose of 0.3 mg/kg dexamethasone for 5 days. Rabbits were monitored for signs of respiratory distress respiratory. If stridor and nasal flaring were noted, rabbits airway was evaluated under sedation using a 0° rigid endoscope (Karl-Storz, Germany). If necessary, tracheal secretions were suctioned and fibrotic-stenotic segments dilated using rubber dilators. Based on the postoperative results of group 1, which included flap edema with tracheal obstruction, the surgical technique was slightly modified for the following 2 rabbits (group 2). One side of the sternohyoid muscle flap was resected prior performing the end-to-end anastomosis in order to improve its venous drainage, which resulted in a partially denuded neotracheal cartilage. Both rabbits experienced endotracheal scarring with stenosis. In order to allow a smooth, vascularised, fibrous capsule to form over the denuded cartilage, in the remaining 2 rabbits (group 3) the silicone tube was re-inserted after the partial resection of the muscle flap. Tracheal reconstruction was performed 6 weeks later.

### Macroscopic Assessment and Histology

Tracheas were harvested, fixed in formalin, embedded in paraffin, cut into 5 micron thick sections, and representative slides stained with Hematoxylin-Eosin and Safranin-O. The specimens were examined on a bright field microscope (Leika DM6000B, Germany) and images were recorded.

### Results

### Clinical Findings

A total of 6 rabbits underwent multi-step tracheal reconstruction, which included fabrication and implantation of engineered neotracheas for 12 to 14 weeks in vivo, followed by segmental tracheal reconstruction. None of the rabbits' neotracheas showed signs of a wound infection and there were no signs of rejection. All neotracheal frameworks maintained their stability and mechanical integrity throughout with no change in shape or size. All 6 animals expired between 1 and 39 days due to tracheal obstruction for the following reasons: rabbits of group I (n=2), experienced venous obstruction of the muscle flap with edema and tracheal obstruction, and died after 24 hours. Rabbits of group II survived 12 and 39 days, and rabbits of group III 14 and 29 days. All 4 rabbits of groups II and III revealed intraluminal fibrosis with a cicatricial stenosis as determined endoscopically (FIG. 8).

### Histology

Externally, no signs of acute or chronic inflammation were detected in any of the 6 specimens. A fibrous capsule containing multiple capillaries and larger blood vessels surrounding the neotracheas externally had formed. The engineered cartilage had undergone a considerable remodeling process as the cartilage layers had integrated into each other with a C-shaped tracheal framework up to 2.5 mm thick. In some areas of the engineered neotracheas cartilage looked much like native auricular cartilage with a typical organization of chondrocytes and healthy cartilage. Safranin O staining indicated that the engineered neotracheal cartilage contained less extracellular glycosaminoglycan than native tracheal cartilage. Some smaller areas of the neotracheal framework revealed hypertrophic chondrocytes. There were also areas, where cartilage had turned into bone. In group I, the muscle flap appeared to be edematous resulting in a complete lumen obstruction. In groups 2 and 3, the muscle flap was viable and intact. Fibrous tissue had developed circumferentially within the lumen resulting in a cicatricial stenosis with its point of maximum in the center of the neotrachea. Respiratory mucosa had migrated into the neotracheal lumen transitioning into a thin layer of non-keratinizing epithelium towards the center of the neotracheal lumen.

### EXAMPLE 3

### Use of Bioreactor to Prepare Autologous Chondrocyte-Driven Repair of Large Bone Defects

Our laboratory has recently shown that chondrocytes grown from ear cartilage are capable of stimulating endochondral bone formation on a large scale. Furthermore, the geometry of this bone formation can be controlled utilizing a silicone template wrapped in these cartilage sheets. Based on those results, autologous ear cartilage can be used as a template to produce cylinders of bone via endochondral bone formation for the repair of large segmental skeletal defects.

Using cell culture and bioreactor methodologies already developed and described in example 1, rabbits are implanted with sheets of ear cartilage wrapped around a silicon tube template placed proximal to the femur and, once bone formation is confirmed by microCT, the vascularized flap of bone is implanted into a segmental defect and held in place with an intermedulary nail. The rabbits are then assessed for bone repair by microCT over time (3, 6, 9 and 12 months) at which time they are sacrificed and assessed for biomechanical strength, and by histomorphometry.

BMP pre-treatments and co-seeding of cartilage sheets with MSCs can be used to increase the rate of bone formation. Sheets of ear and articular cartilage are exposed to varying amounts of BMP's 2, 6 and 7 and can be assessed for the rate of endochondral bone formation, in vivo, by microCT.

Having described the invention, we claim:

1. A bioreactor comprising:
a housing including a member having oppositely disposed first and second surfaces and an inner surface defining an opening, the opening extending between the first and second surfaces of the member;
a gas and liquid permeable membrane having first and second surfaces attached to the second surface of the member, the first surface of the gas and liquid permeable membrane and the member defining a culture space for growing or culturing cells;
a culture vessel capable of receiving the housing and having a volume defined by a bottom surface and at least one side wall, the culture vessel including a serum-free culture medium; and
a support mechanism for suspending the housing above the bottom surface of the culture vessel so that the serum-free culture medium can contact the second surface of the gas and fluid permeable membrane.

2. The bioreactor of claim 1, the housing including a second member having oppositely disposed first and second surfaces and an inner surface defining an opening, the opening extending between the first and second surfaces of the second member.

3. The bioreactor of claim 2, the gas and liquid permeable membrane being sandwiched between the second surface of the member and the first surface of the second member.

4. The bioreactor of claim 1, the securing mechanism comprising at least one member having first and second ends, the second end of the at least one member contacting the bottom surface of the culture vessel to support the housing so that the serum-free culture medium can contact the second surface of the gas and fluid permeable membrane.

5. The bioreactor of claim 1, the culture vessel comprising a Petri dish.

6. The bioreactor of claim 1, the housing being positioned in the culture vessel such that the first surface of the member is elevated above the surface of the serum-free culture medium.

7. A method for generating a cartilage tissue construct, the method comprising the steps of:
isolating a population of chondrogenic cells;
expanding the population of chondrogenic cells;
seeding the population of chondrogenic cells into a bioreactor, the bioreactor comprising a housing including a member having oppositely disposed first and second surfaces, an inner surface defining an opening extending between the first and second surfaces, a gas an liquid permeable membrane having first and second surfaces attached to the second surface of the member, the first surface of the gas and liquid permeable membrane and the member defining a culture space for growing or culturing the population of chondrogenic cells, a culture vessel having a volume defined by a bottoms surface and at least one side wall and including a serum-free medium, and a support mechanism for suspending the housing above the bottom surface of the culture vessel; and
culturing the population of chondrogenic cells in the culture space of the bioreactor for a time sufficient to permit the population of chondrogenic cells to differentiate and form the cartilage tissue construct.

8. The method of claim 7, the step of expanding the population of chondrogenic cells further comprising culturing the population of chondrogenic cells in a conditioned growth media effective to promote expansion of the chondrogenic cell population.

9. The method of claim 7, the population of chondrogenic cells being autologous, allogeneic, or a combination thereof.

10. The method of claim 7, the population of chondrogenic cells being immature chondrocytes, mature chondrocytes, or a combination thereof.

11. The method of claim 7, the population of chondrogenic cells endogenously producing an extracellular matrix when the population of chondrogenic cells is cultured in the culture space of the bioreactor.

12. The method of claim 7, the serum-free culture medium including at least one growth factor selected from the group consisting of transforming growth factor-, platelet-derived growth factor, insulin-like growth factor, acid fibroblast growth factor, basic fibroblast growth factor, epidermal growth factor, hepatocytic growth factor, keratinocyte growth factor, and bone morphogenic protein.

13. The method of claim 7, the step of culturing the population of chondrogenic cells in the bioreactor comprising growing the population of chondrogenic cells at about 37° C. in a humidified atmosphere with the addition of about 5% carbon dioxide and about 1% to about 21% oxygen.

14. The method of claim 7, the cartilage tissue construct having a thickness of about 200 microns to about 4 mm.

15. A cartilage tissue construct produced by the method of claim 7.
